(19) United States
(12) Patent Application Publication
(10) Pub. No.: US 2022/0165412 A1
(43) Pub. Date: May 26, 2022

(54) APPARATUS AND METHOD FOR MONITORING AND ASSISTING PREGNANT WOMEN

(71) Applicants: Hyunjeung IN, Germantown, MD (US); Sungyong IN, Germantown, MD (US)

(72) Inventors: Hyunjeung IN, Germantown, MD (US); Sungyong IN, Germantown, MD (US)

(21) Appl. No.: 17/533,057

(22) Filed: Nov. 22, 2021

Related U.S. Application Data
(60) Provisional application No. 63/116,915, filed on Nov. 22, 2020.

(51) Int. Cl. G16H 40/67; G16H 50/30; G16H 50/20; G16H 20/10; A61B 5/00

(52) U.S. Cl. CPC G16H 40/67; G16H 50/30; A61B 5/681; G16H 20/10; A61B 5/4343; G16H 50/20

(57) ABSTRACT

A system for providing health services to a pregnant woman away from a medical facility is provided. The system may be configured to include first and second wearable devices to be worn by the pregnant woman. The first wearable device may be configured to collect a first set of health data of the pregnant woman and the second wearable device may be configured to collect a second set of health data of an unborn baby growing in the pregnant woman. Further, the system may be configured to include a mobile device configured to: communicate with the first and second wearable devices; collect the health data of the pregnant woman and her unborn baby; and communicate with a remote hospital personnel via one or more servers over a cloud-based network. The mobile device may be further configured to receive guidance or instructions from the remote hospital personnel in response to transmission of the health data of the pregnant woman and her unborn baby.

### CROSS-REFERENCE TO RELATED APPLICATION(S)

This application claims the benefit of U.S. Provisional App. No. 63/116,915, filed on Nov. 22, 2020, which is incorporated herein by reference in its entirety.

### TECHNICAL FIELD

The present disclosure relates to method and apparatus for monitoring and/or assisting people away from a hospital and, in particular, for monitoring and/or assisting pregnant women away from a medical facility during their pregnancy.

### BACKGROUND

Pregnancy brings joy to many including soon would-be mothers, but at the same time uncertainties and worries to the soon would-be mothers. There exist many websites and portals relating to pregnancy, providing many useful information such as what-to-do list for pregnant women and body changes they are about to go through. However, many of the websites often lack accurate medical information and provide piece-wise information in some areas.

Pregnancy information may be spread over many different websites and portals and as a result people often tend to pour many hours into reviewing various pieces of information on the websites. Some websites may focus on certain aspects of pre-natal education resources and materials, some websites may provide information on pre-natal drugs or supplement products, and other websites may provide what to do and what not to do, based on their own experiences. As mentioned, there is a lack of unified, single portals for the pre-natal and pregnancy related information that can be easily obtained and available.

Further, women often find out their pregnancy by noting some changes in their body conditions or by performing pregnancy tests at home or medical facility. After that, they will make regular scheduled visits to a hospital or medical facility for consultation with doctors (e.g., obstetricians) and/or nurses and periodic check ups. Traditionally, after the initial consultation, the pregnant women will make periodic scheduled visits (e.g., once a month) during their trimester periods. However, there is no easy way to monitor and communicate with the hospital and doctors during a time between these regular scheduled visits. In most cases, the pregnant women will obtain medically accurate information from their doctors or medical professionals at the hospital or medical facility only during their regular scheduled visits. Often, the pregnant woman worries about certain bodily changes such as morning sickness, back and leg pain, heartburn, and other common pregnancy complaints.

As such, there is still a need for having more advanced technologies for pregnant women and hospital staff to do real-time communications between the pregnant women and the hospital, for ensuring and maintaining healthy pregnancy and well-being of soon would-be mothers and their children. Further, there is still a need for advanced mobile platforms for helping the pregnant women to better cope with their changes, as well as capture many memorable moments or events, for future purposes, thereby increasing the joy of having pregnancy without having worries and concerns about their body changes during their pregnancy.

### SUMMARY

The present disclosure or technology described herein provides a method or system for providing enhanced health services for pregnant women. By way of example, the system may be configured to include a first wearable device, a second wearable device, an electronic scale, and a mobile device. The first wearable device may be worn by the pregnant woman and configured to collect a first set of health data of the pregnant woman. The second wearable device may be worn by the pregnant woman and is configured to collect a second set of health data of an unborn baby growing in an abdomen of the pregnant woman. The mobile device may be configured to communicate with the first wearable device and the second wearable device. Further, the mobile device may be configured to receive the health data of the pregnant woman and the unborn baby from the first, second wearable devices as well as the electronic scale, and to communicate with remote hospital staff over a cloud based network. Also, in response to transmission of information on the health data of the pregnant woman and the unborn baby, the mobile device may be configured to receive guidance or instructions from the remote hospital staff.

In an aspect of the present disclosure, the first set of health data may include health data of the pregnant woman such as her heart rate, blood pressure, weight, glucose level, body temperature, respiratory rate, and/or oxygen saturation level, and the second set of health data may include health data of her fetus or unborn baby such as a heart rate, body parts, measurements, or size of the unborn baby.

In another aspect of the present disclosure, the first wearable device may include a smart watch or a smart band that is to be worn on a wrist of the pregnant woman.

In another aspect of the present disclosure, the second wearable device may include a smart device disposed on an abdomen area of the pregnant woman for collecting health data of the fetus or unborn baby growing inside the pregnant woman.

In an aspect of the present disclosure, the mobile device may be configured to present data analysis information to the pregnant woman, based on analysis of the health data collected from the pregnant woman.

Further, in another aspect of the present disclosure, the mobile device may be configured to receive guidance or instructions from the hospital staff, in response to the transmission of the health data of the pregnant woman and her baby to the remote hospital.

In another aspect of the present disclosure, the mobile device may also include one or more artificial intelligence (AI) agents for analyzing the health data collected from the pregnant woman and presenting one or more recommendations to the pregnant woman based on the analysis of the collected health data.

In an aspect of the present disclosure, the electronic scale may be configured to measure a body weight of the pregnant woman and other health data and may be configured to wirelessly communicate with the mobile device of the pregnant woman.

In another aspect of the present disclosure, the mobile device may be configured to provide the pregnant woman with various educational information including tips relating to pregnancy, based on the collected health of the pregnant woman.

In an aspect of the present disclosure, the mobile device may be configured to receive one or more messages from the remote hospital personnel when an abnormality is detected in the health data of the pregnant woman and her baby.

In an aspect of the present disclosure, the mobile device may be configured to automatically provide the pregnant woman with one or more recommendations for dietary meals for the pregnant woman, based on a profile and the health data of the pregnant woman.

In another aspect of the present disclosure, the mobile device may also be further configured to provide the pregnant woman with one or more recommendations for post-delivery care facilities based on the profile and the health data of the pregnant woman.

In another aspect of the present disclosure, the mobile device may be further configured to provide the pregnant woman with one or more recommendations for supplementary drugs, medicines or products, based on the profile and the health data of the pregnant woman.

In another aspect of the present disclosure, the system of the present technology may be further configured to produce a digital record book relating to the pregnancy of the woman.

In another aspect of the present disclosure, the digital record book may include digital pictures, photos, audios, writings, or any combinations thereof, which are inputted by the pregnant woman.

### BRIEF DESCRIPTION OF THE DRAWINGS

A more detailed understanding may be obtained from the following description in conjunction with the following accompanying drawings.

FIG. 1 illustrates an example embodiment of the present technology in accordance with an aspect of the present disclosure;

FIG. 2 is a diagram conceptually illustrating an example system in accordance with an aspect of the present disclosure;

FIG. 3 is another diagram conceptually illustrating an example embodiment of the present technology in accordance with an aspect of the present disclosure;

FIG. 4 is an exemplary table illustrating typical values of certain vital signs for pregnant women in an aspect of the present disclosure;

FIG. 5 is another diagram conceptually illustrating example embodiments of the present technology in accordance with an aspect of the present disclosure;

FIG. 6 illustrates an example embodiment of user interfaces on a mobile device in accordance with an aspect of the present disclosure;

FIG. 7 is a diagram conceptually illustrating an exemplary embodiment of the present technology in accordance with an aspect of the present disclosure;

FIG. 8 is a diagram conceptually illustrating an exemplary embodiment of the present technology in accordance with an aspect of the present disclosure;

FIG. 9 is a diagram conceptually illustrating an exemplary embodiment of the present technology in accordance with an aspect of the present disclosure;

FIG. 10 is another diagram conceptually illustrating an exemplary embodiment of the present technology in accordance with an aspect of the present disclosure; and

FIG. 11 is another diagram conceptually illustrating an example embodiment of the present technology in accordance with an aspect of the present disclosure.

### DETAILED DESCRIPTION

The detailed description of illustrative examples will now be set forth below in connection with the various drawings. The description below is intended to be exemplary and in no way limit the scope of the claimed invention. It provides detailed examples of possible implementation(s), and as such they are not intended to represent the only configuration(s) in which the concepts described herein may be practiced. That is, the detailed description includes specific details for the purpose of providing a thorough understanding of various concepts, and it is noted that these concepts may be practiced without these specific details. In some instances, well known structures and components are shown in block diagram form in order to avoid obscuring such concepts and like reference numerals are used in the drawings to denote like elements and features.

It is also noted that in some instances while the methodologies are described herein as a series of steps or acts, for the purpose of simplicity it is to be understood that the claimed subject matter is not limited by the order of these steps or acts, as some steps or acts may occur in different orders and/or concurrently with other acts from that shown and described herein. Further, not all illustrated steps or acts may be required to implement various methodologies according to the present technology disclosed herein. Also, it should be appreciated that the apparatus and methods described herein may be utilized separately or in combination with other aspects of the present disclosure, or in combination with conventional technology, without departing from the teachings of the present disclosure.

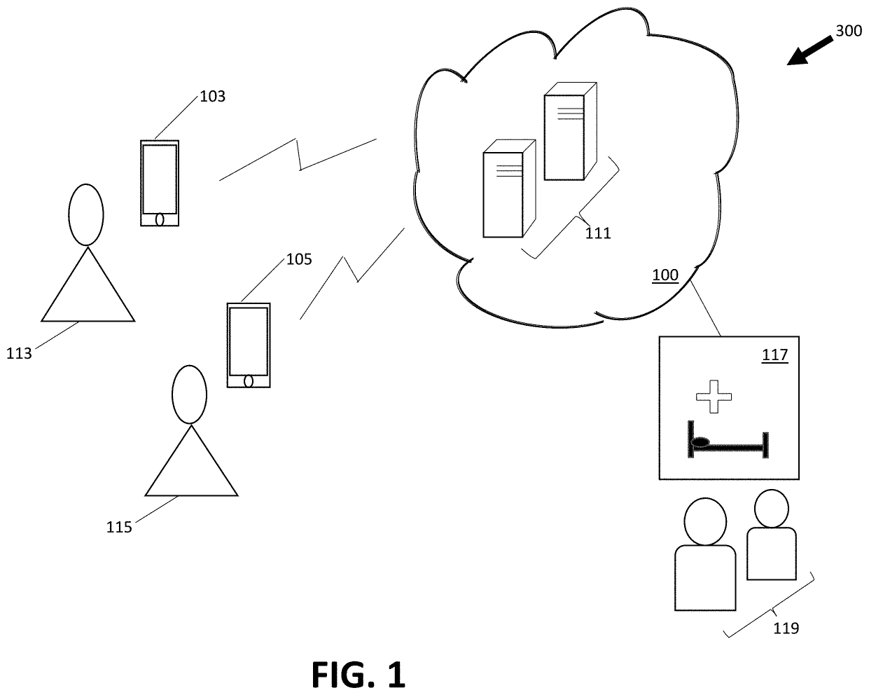

FIG. 1 is a high-level diagram of a system or apparatus conceptually illustrating an embodiment of the present technology. In the system, a mobile device is used to collect health data related to the pregnancy of a woman and is used in communicating with doctors and staff at a hospital over a cloud network. Typically, a pregnant woman will visit doctors at a hospital and during her visit(s) to the hospital, many medical testing is conducted, for example, blood testing, measurements of blood pressure, weight, glucose levels, etc. to obtain a medical baseline for the pregnant woman to provide proper pregnancy care. After her first hospital visit, the pregnant woman generally makes subsequent scheduled visits with the doctors and staff during her pregnancy. The scheduled visits are, however, based on general pregnancy schedules as well as availability of the hospital and doctors' schedules. Between those scheduled visits, however, certain unexpected situation may develop with the pregnant woman and she may not receive prompt, proper medical attention or counseling to help them ease their concerns. Sometimes, pregnant women may miss critical time for taking any remedial actions to avoid pregnancy complications. That is, although the pregnant women may have needs, all parties involved may fail to take any proactive measure or timely actions against the development of certain pregnancy emergency situations or complications. Further, outside the scheduled hospital visits, the doctors and hospital staff may not have any good, established means for communicating with their patients or pregnant women for monitoring and/or managing the pregnancy. As such, the present technology disclosed herein provides, among other things, advanced systems or apparatuses for prompt communications, monitoring, management of the pregnancy, as well as for early responses to any developing pregnancy situation for the pregnant women and the hospitals or doctors.

By way of example, an embodiment of a system 300 of FIG. 1 is configured to address some of these needs of the pregnant women and their doctors or hospital personnel. The system 300 is configured or designed to receive or collect certain health data including vital data from the pregnant women (e.g., patients 113, 115), and store them either locally (e.g., mobile devices 103, 105) and/or on the cloud-network (e.g., one or more servers 111) for the purpose of processing the collected data, or send them over the cloud-network 100 to their doctors and/or staff 119 at the hospital or medical facility 117. Then, the doctors and/or staff 119 may receive and review the health data as well as take any proactive actions based on the analysis of the collected health data from the pregnant women.

In one implementation, the system 300 of FIG. 1 may include at least a mobile device with one or more mobile applications. In an aspect of the present disclosure, the pregnant woman 115 may download one or more mobile applications 120 (e.g., mobile pregnancy health applications) to implement one or more aspects of the present disclosure. The mobile applications 120 may be downloaded from the one or more servers 111 or certain web application stores such as app stores (e.g., Apple app store, Google app store, Windows app store, or the like). Further, in an aspect of the present disclosure, the one or more mobile applications 120 along with other devices, such as data collection devices 123, 125, 131, and the hospital or medical facility 117 may form an integrated platform for providing health services relating to pregnancy of women.

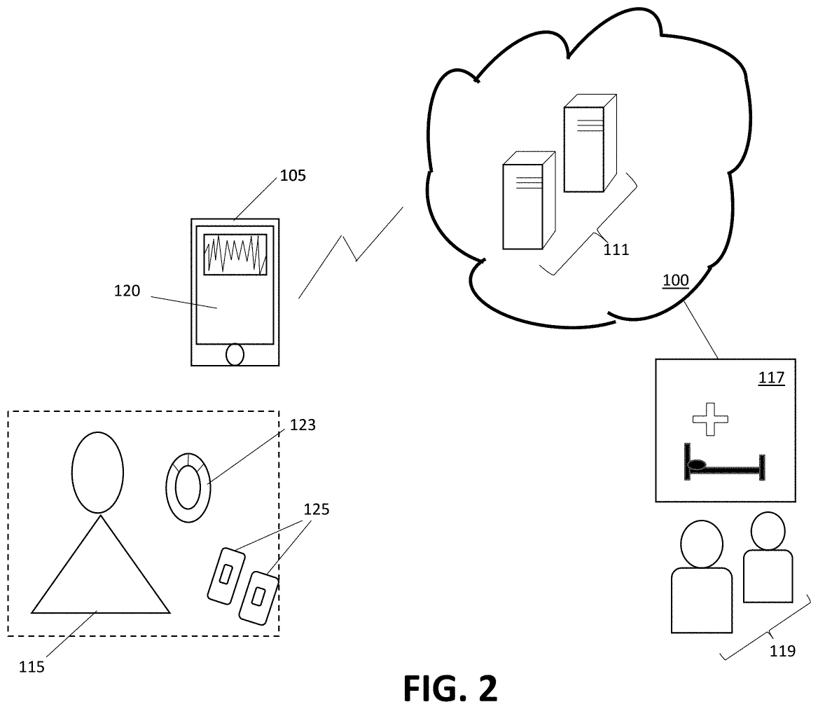

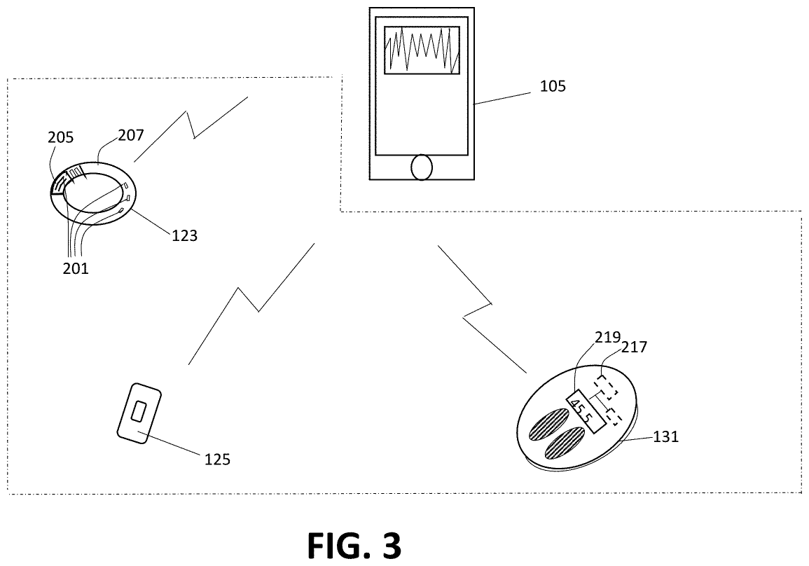

FIG. 2 illustrates exemplary data collection devices as well as a mobile device configured to communicate with the exemplary data collection devices. The exemplary data collection devices may include the wearable devices 123, 125 as well as the electronic scale 131 as shown in FIG. 3. By way of example, the mobile device 105 (which includes mobile applications 120) may be configured to communicate with the first wearable device 123, the second wearable device 125 and the electronic scale 131 for collecting health data of the pregnant women 115 and her fetus or unborn baby. It is noted that although the present technology is illustrated for the pregnant women, the present technology may not be limited thereto and may include patients who may need remote health monitoring and services from the remote hospital or medical facilities.

In an aspect of the present disclosure, in the example, the first wearable device 123 may be configured to collect the vital health data of the pregnant woman 115 (e.g., vital pregnancy data) and the second wearable device 125 may be configured to collect vital health data of a fetus or an unborn baby growing inside the pregnant woman 115. The mobile device 105 may further be configured to provide control and management functions of the data collection devices including the first, second wearable devices 123, 125 and the multi-functional electronic scale 131. In one implementation, the mobile device 105 may be in the form of a smart phone, a tablet, or any other computing device that may be configured to wirelessly communicate with the first wearable device 123, the second wearable device 125 and the electronic scale 131 as shown in FIGS. 2 and 3.

In another aspect of the present disclosure, the mobile device 105 may be configured to operate as an integrated platform for collecting health data from the pregnant woman and communicating with the hospital 117 as well as hospital doctors or staff 119, in accordance with various aspects of the present disclosure.

As mentioned above, in one implementation, the first wearable device 123 may be configured to collect the health data related to the pregnant woman 115, such as her body temperature, blood pressure (e.g., systolic blood pressure and/or diastolic blood pressure), blood glucose, oxygen saturation SpO2, respiratory rate, etc. which are generally considered as vital statistics for woman's pregnancy. To collect these vital health data, the first wearable device 123 may be in the form of a smart watch, a wrist band, a patch type, or any other devices that can be worn by the pregnant woman 115 so that the woman's health data during the pregnancy may be collected. Also, the data collection may be performed continuously or certain periodic intervals. This periodic data collection interval may be set by the hospital, medical facility or doctors. Further, in addition to the vital pregnancy data, other health data may be collected from the pregnant woman via the data collection devices 123, 125, 131.

As mentioned above, the first wearable device 123 may be configured to collect, among many others, health data relating to the pregnancy of the woman, such as her heart rate, blood pressure, blood glucose, temperature, oxygen saturation level, etc. relating to the pregnant woman. The second wearable device 125 may be configured to collect, among many others health data relating to the fetus or unborn baby growing inside the woman, such as the baby's heart rate, size, measurements, other images, etc. The electronic scale 131 may be configured to collect her weight, body mass index (BMI), etc. Further, the mobile device 105 may receive all the collected health data from the first wearable device 123, the second wearable device 125, and the electronic scale 131.

In another aspect of the present disclosure, the first wearable device 123 may be configured to include at least a display 205, a wrist band 207, and one or more sensors 201 on the contact surface of the wrist band 207. The one or more sensors 201 may be configured to collect the health data of the pregnant woman, such as blood pressure, blood sugar level, heart rate, etc. Also, the first wearable device 123 may be configured to wirelessly communicate with the electronic scale 131, via wireless communications technology such as Bluetooth, WiFi, near field communications, or any other wireless communications technology. Alternatively, the mobile device 105 may be configured to wirelessly communicate with the first wearable device 123, the second wearable device 125, and the electronic scale 131, to receive the health data of the pregnant woman 115, such as blood pressure, blood sugar level, heart rate, weight, BMI, etc., through wireless communications technology such as Bluetooth, WiFi, near field communications, or other wireless communications technology. Also, the electronic scale 131 may be configured to collect data such as the pregnant woman's weight, BMI data, etc. as she steps on the electronic scale 131. That is, the first wearable device 123, the second wearable device 125 and the electronic scale 131 may be configured to include one or more wireless transceivers for wireless communications with the mobile device 105 or other computing devices.

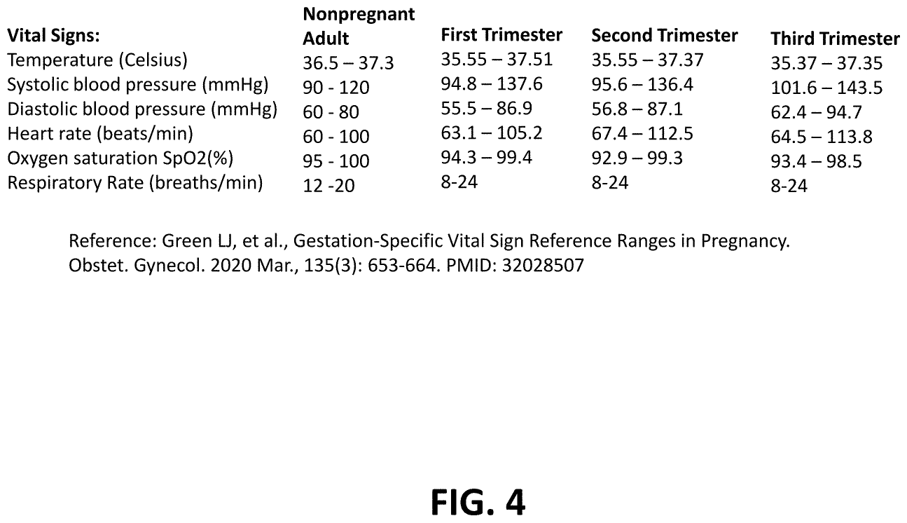

FIG. 4 is a table conceptually illustrating exemplary vital pregnancy data in general, which may be used as one or more reference frameworks for the pregnant women in accordance with an aspect of the present disclosure. In the example, during the first trimester of the pregnancy, the normal temperature is observed to have a range of 35.55-37.51 degrees (in Celsius), the normal systolic blood pressure is observed to have a range of 94.8-137.6 mmHg, the diastolic blood pressure is observed to have a range of 55.5-86.9 mmHg, the normal heart rate is observed to have a range of 63.1-105.2 beats/minute, and the normal oxygen saturation is observed to have a range of 94.5-99.4%. During the second trimester of the pregnancy, it is noted that the normal temperature is observed to have a range of 35.55-37.37 degrees (in Celsius), the normal systolic blood pressure is observed to have a range of 95.6-136.4 mmHg, the diastolic blood pressure is observed to have a range of 56.8-87.1 mmHg, the normal heart rate is observed to have a range of 67.4-112.5 beats/minute, and the normal oxygen saturation is observed to have a range of 92.9-99.3%. During the third trimester of the pregnancy, it is noted that the normal temperature is observed to have a range of 35.37-37.35 degrees (in Celsius), the normal systolic blood pressure is observed to have a range of 101.6-143.5 mmHg, the diastolic blood pressure is observed to have a range of 62.4-94.7 mmHg, the normal heart rate is observed to have a range of 64.5-113.8 beats/minute, and the normal oxygen saturation is observed to have a range of 93.4-98.5%. As mentioned above, these and other data may be used as reference data for vital health data for the purpose of comparing and analyzing the health data collected from the pregnant women 113, 115 during their pregnancy as shown in FIGS. 1 and 2 in accordance with various aspects of the present disclosure.

As mentioned above, the mobile device 105 may act as a central device configured to wirelessly receive maternal heath data transmitted from the first wearable device 123, the health data of the fetus or unborn baby transmitted from the second wearable device 125, and additional health data of the pregnant woman from the multifunctional electronic scale 131. The mobile device 105 may also be configured to perform data analytics on the received health data for gleaning insights and monitoring the progress of the pregnancy. In another aspect, the mobile device 105 may be configured to store the received or collected health data in its storage, or alternatively send them to one or more cloud storages and/or servers disposed at the could or at the hospital or medical facility, for the purpose of data storage and performing data analytics. That is, the mobile device 105 may be configured to transmit one or more portions or all the collected health data from the pregnant woman 115 to a cloud service and/or the hospital or medical facility 117, which the pregnant woman 115 has scheduled visits for receiving her pregnancy health care during and/or after her pregnancy. Further, at the hospital or medical facility 117, doctors, staff or personnel 119, who are supposed to provide medical care of the pregnant woman 115, may access the health data of the pregnant woman 115 and review them for any presence of abnormality in the collected health data, aided by the one or more AI agents working on the collected health data. When the presence of any abnormality is detected, the doctors, staff or personnel 119 may be notified so that they can take prompt, direct actions that are needed as preventive measures against any possible complication or other proactive actions to improve the situation.

By way of example, the doctors who have access to the health data of the pregnant woman via the present technology disclosed herein may review the health data, such as the mother's blood pressure, weight, blood glucose, heart rate, fetal heart rate or any other vital health measurements of the fetus, which have been collected through data collection devices including the first wearable device 123, the second wearable device 125, and the electronic scale 131. As noted above, the one or more AI agents may also review the health data and detect any presence of any abnormality. When any abnormality in the health data is detected or there is any trend which may cause any concern for the well-being of the pregnant woman and/or the unborn baby, the one or more AI agents may generate alerts to the doctors or staff at the hospital or the medical facility 117 who may initiate direct communications with the pregnant woman 115. For example, the collected health data may be analyzed for health conditions that may cause problems during the pregnancy or affect the baby's health such as high blood pressure, diabetes, infections, genetic disorders, etc. When an abnormality is detected, the mobile device 105 (or e.g., a mobile application 120) may generate one or more alerts to the pregnant woman 115 and/or the hospital staff or personnel 119 and in response receive instructions from the hospital staff or personnel 119 for further actions. In the example, a text alert message or a contact alert message may be generated and displayed or through audible notes and one or more text alerts or messages may also be left in a communication window of the mobile application 120 so that the pregnant women may review them on their mobile devices.

Referring back to FIG. 3, in another aspect of the present disclosure, the mobile device 105 may be configured to receive and analyze the data from the first wearable device 123, the second wearable device 125, and the electronic scale 131. In other words, the mobile device 105 may act as a central device configured to receive and combine various data relating to the health conditions of the pregnant woman and the fetus or unborn baby. Further, using the one or more AI agents running on the mobile device 105, the mobile device 105 may analyze the combined health data, prior to transmitting them to the hospital or medical facility 117. That is, the mobile device 105 may be configured to analyze the collected data and may present one or more recommendations to the pregnant woman for any action or informational purposes, via one or more user interfaces of the mobile device 105. Alternatively, the collected health data may be sent by the mobile device 105 to the one or more servers 111 on the cloud-network for the purpose of performing analysis of the health data and determining one or more recommendations to the pregnant woman as well as to the hospital or medical facility 117 or doctors and staff 119 for their review and action. Also, in another implementation, the one or more servers 111 may include the one or more AI agents for the analysis of the collected health data and determining the one or more recommendations. In an aspect of the present disclosure, depending upon the amount of data and processing power available, the storage and the analysis of the collected data may be performed at different devices such as at mobile devices 105, at servers on the cloud 111, or at computing devices at hospital or medical facility 117.

In an aspect of the present disclosure, for the analysis of the collected health data from the pregnant women, typical vital health data relating to a normal pregnancy may be used as well as other data. The collected vital health data of the pregnant woman 115 may be combined with other health data to form big data for the purpose of monitoring and analysis. In one implementation, comparing with the other data on normal healthy pregnancy, the collected health data of the pregnant woman 115 and her unborn baby may be monitored and analyzed for any proactive actions to be taken, in case of an occurrence of certain deviations from the normal, healthy pregnancy data. That is, the collected vital health data such as the woman's temperature, blood pressure, heart rate, oxygen saturation level, and respiratory rate may be compared against the typical vital health data for a normal pregnancy, as shown in FIG. 4; that is, it is determined whether or not the collected data fall in the acceptable ranges for the typical pregnancy, as shown in FIG. 4. Further, it is noted that based on the big data including data from other pregnant women, the normal, healthy pregnancy data may be grouped into multiple classification groups, depending upon a different profile of pregnant women, such as ages, colors of skin, ethnic groups, pre-existing conditions, countries, geographical regions, etc. and the health data collected from the pregnant woman 115 may be compared with the normal ranges of the different classification groups (or reference data) for more precise monitoring and analysis purposes, which is selected based a profile of the pregnant woman 115.

In an aspect of the present disclosure, as mentioned above, the vital pregnancy health data from the subject pregnant woman and other collected health data from other women may be combined to become part of reference data in monitoring, analyzing, and determining the status of the pregnancy of the woman 115. That is, the statistical vital data from other pregnant women may be used as big data reference and used to detect any abnormality in the pregnancy of the pregnant women 113, 115, based on the collected health data from the pregnant women 113, 115. As such, based on the combined big data reference, the mobile device 105 may be configured to perform the data analytics on the health data collected from the pregnant woman 115 and determine whether the health data collected from the pregnant woman 115 are in the typical ranges of the big data reference, or determine whether the pregnancy is normally progressing.

Further, the big data reference may be stored in local devices such as mobile device 105, or at the cloud such as the one or more servers 111. Alternatively, the big data reference may be stored in one or more computers on the premises of the hospital or medical facility 117. The storage of the big data as well as collected health data may not be limited to a single location, but rather may be at different locations or distributed over the web, depending upon the computing capability and design architecture of the system disclosed herein.

In an aspect of the present disclosure, when the health data of the pregnant woman 115 is compared with the big data reference, any abnormality detected may trigger or send one or more alerts to the hospital or medical facility for their review of the collected health data from the pregnant woman 115 and her unborn baby. For example, the hospital or medical facility or staff may review and analyze the collected health data for any health conditions that may cause problems during the pregnancy or affect the baby's health such as high blood pressure, diabetes, infections, genetic disorders, etc. When the detected abnormality is determined to be serious and need further professional attention, the hospital or medical facility 117 or hospital staff 119 may contact the pregnant woman 115 for further consultation and check-ups at the hospital or medical facility 117, prior to originally scheduled regular appointments. That is, upon the review of the health data and any alerts generated, the hospital personnel or staff 119 may immediately contact the pregnant woman 115 through various means including the mobile device 105, phones, emails, texts, etc. for consultation and check-ups, thereby initiating prompt communications and taking proactive actions before it develops into a more serious situation. As such, the present technology disclosed herein provides advanced means and technology for serving as an early warning system as well as an integrated communications platform for providing personalized, prompt medical services to pregnant women, thereby preventing any pregnancy complication as early as possible.

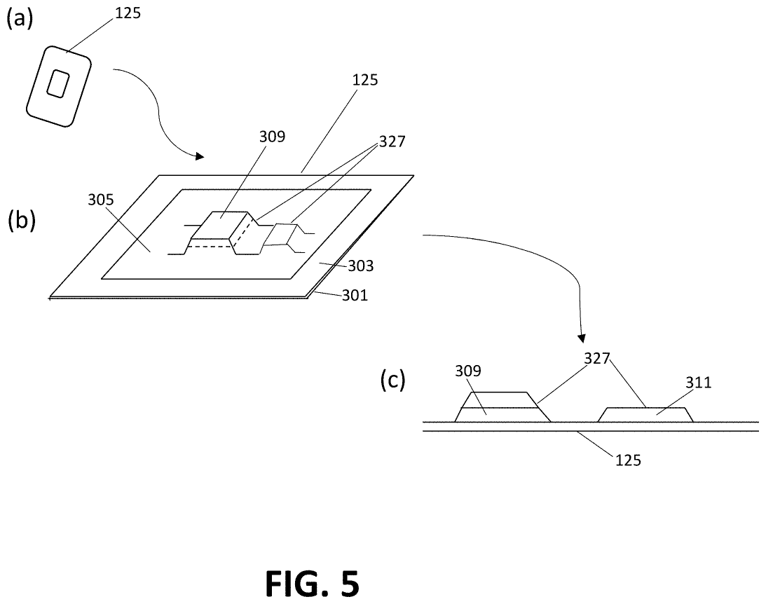

FIG. 5 illustrates exemplary embodiments of the second wearable device 125 as shown in FIG. 3. In an aspect of the present disclosure, the mobile device 105 (e.g., or alternatively the mobile application 120) may be configured to wirelessly communicate with the second wearable device 125 to collect or receive the health data of the fetus or unborn baby growing in the abdomen area of the pregnant woman 115. The second wearable device 125 may be in the form of a patch as shown in FIG. 5(a), which may be adhered to or disposed on an area of the abdomen of the pregnant woman 115 such that the second wearable device 125 may be configured to collect the health data of the fetus or unborn baby growing inside the abdomen of the pregnant woman 115. It is also noted that the implementation of the second wearable device 125 may not be limited to the disclosed forms but may be implemented in other forms and designs including various forms and designs. By way of example, in one implementation, the second wearable device 125 may be a medical patch with enhanced functions for data collection. Further, the second wearable device 125 may include one or more small electronics or electronics devices 327 for performing various functions, as shown in FIG. 5(b), such as monitoring and collecting health data relating to a fetus or baby growing in an abdomen of a pregnant woman, and the health data collected may include the fetus's heart rate, position, size dimension, locations, etc.

Further, the second wearable device 125 may include small scale electronics capable of performing, supporting or implementing various technology, as shown in FIGS. 5(a)-(c), ultrasound imaging technology, acoustic imaging technology, or variations thereof, and/or other technology which may be used in imaging or detecting the fetus or unborn baby and collecting health data on the fetus or unborn baby. Furthermore, the second wearable device 125 may be configured to work with the mobile device 105 that may perform certain functions such as image processing or rendering of the images of the fetus or unborn baby in the pregnant woman 115 based on the received data from the second wearable device 125.

Further, the second wearable device 125 may be implemented in a plurality of wearable devices which may be configured to collect various measurement data relating to the fetus or unborn baby. By way of example, the plurality of wearable devices may be used to collect data on the fetus or baby, such as presence and size dimensions of arms, legs, and other body parts, head and brain, heart chambers and valves, kidneys, placenta placement, spine, etc., and the collected data may be used by the mobile device 105 (or one or more AI agents) in determining whether or not the fetus or unborn baby is normally growing in the pregnant woman. Also, the collected data may be used by the one or more AI agents to determine whether there is any signs of a possible pregnancy complication.

Referring back to FIG. 5, the second wearable device 125 shown in FIG. 5(a) may be configured to include one or more micro or nano scale electronics configured to perform various functions, such as ultrasound imaging, measurement, as well as other data collection, as shown in FIG. 5(b). In particular, the patch 125 shown in FIG. 5(b) may include one or more micro- or nano-electronics 327 that may be designed to collect various data concerning the fetus/unborn baby growing in the abdomen of the pregnant woman 115. Alternatively, the patch 125 may be used to collect the health data of the pregnant woman 115. The one or more micro- or nano-electronics 327 may include power circuitry and may be embedded at separate locations of a patch area. In another aspect of the present disclosure, the one or more micro- or nano-electronics 327 may be stacked on top of another, as shown in FIG. 5(c). The arrangement or structure of the patch 125 may depend upon its design architecture.

Further, in another aspect of the present disclosure, the second wearable device 125 may include a patch with enhanced functions such as a miniaturized ultrasonic transducer. In one implementation, the second wearable device 125 may be configured to include at least an adhesive layer 301, a bottom layer 303, a sensor layer 305, a transducer ultrasonic layer 309, and a control layer 327. In the example, the adhesive layer 301 includes non-toxic adhesive gels such that it adheres on the skin of an abdomen area of a pregnant woman. The bottom layer 303 is disposed on top of the adhesive layer 301 and include or support one or more small scale electronics for various functions. The sensor layer 305 may include various sensors for collecting different types of health signals. The transducer ultrasonic layer 309 may include small scale electronics configured to generate ultrasounds and receive echoes of the ultrasounds for detecting and imaging the unborn baby in the abdomen of the pregnant woman and collect data relating to the unborn baby, such as a heart rate, size, location, position orientation, etc. In one implementation, the transducer ultrasonic layer 309 may include one or more active elements made of ceramic crystal materials (e.g., piezoelectric). The transducer ultrasonic layer 309 may include miniature ultrasound transducer(s) to send out a beam of sound waves into the body and the sound waves are reflected back to the transducer by boundaries between tissues in the path of the beam. When these echoes hit the transducers, they generate electrical signals that are sent to the ultrasonic layer 309 and back to the mobile device 105 for processing of the electrical signals for generating two dimensional images of tissues and organs. In an aspect of the present disclosure, the second wearable device 125 may be configured to work with the mobile device 105 to generate the multi-dimensional images of a fetus or unborn baby inside the pregnant woman 115 and to monitor the growth and development of the fetus or unborn baby.

The control layer 327 may include other small scale electronics configured to include certain electronics, processing units, and memory units to store the collected data as well as wirelessly transmit them to the mobile device 105 or the first wearable device 123 or other devices. In the example, the processing units may be configured to process part or all of the collected data into more useful data such as heart rates, sizes, images, or etc. of the unborn baby during the collection intervals.

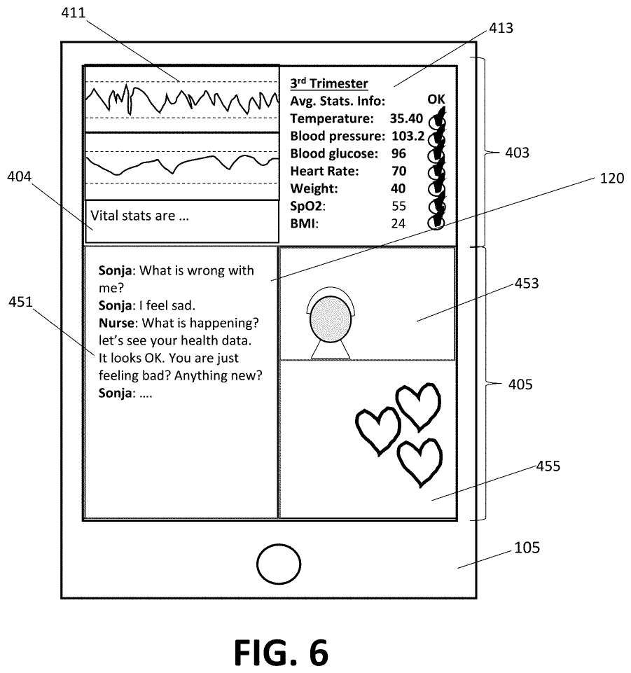

FIG. 6 is a diagram conceptually illustrating an exemplary embodiment of the mobile device 105 along with the mobile application 120 in accordance with various aspects of the present disclosure. The mobile application 120 may be configured to include one or more different views, frames or windows for various purposes. By way of example, the mobile application 120 may be configured to display various pieces of information via one or more user interface frames, e.g., one or more graphical user interfaces 403, 411, 413, 451, 453, 455, to the user of the mobile device 105, e.g., the pregnant woman 115, based on the collected data from a plurality of devices, including the first wearable device 123, the second wearable device 125, the electronic scale 131, or the like.

By way of example, in the example, the mobile application 120 may include at least a front display window comprising various view windows such as data or statistics view 413, communications view 405, etc. The statistics view 403 may be configured to include one or more graphical representations or historical or time-lined displays of the collected data from the pregnant woman 115, an explanation view 404, and a statistics summary view 413. Further, the communications view 405 may include a messaging window 451 configured to support various forms of messaging such as texts, instant messages, emails, etc. and a real-time communications view 453 for video and/or audio calls, as well as other gadgets or applications view 455 (which may be selected and defined by user). In the example, the messaging view 451 and the real-time communications view 453 may be used by the pregnant women 115 and the hospital or medical facility 117 and/or doctors or staff 119 for communications purposes. By way of example, the mobile application 120 may be used to advise the pregnant woman on diet, exercise, medications, and stay healthy during the pregnancy, as well as help coping with morning sickness, back and leg pain, heartburn, and other common pregnancy complaints.

The mobile application 120 or the mobile device 105 may be further configured to exchange various forms of data and messages with the hospital or medical facility 117 or staff 119 such that the pregnant woman 115 receive proper pregnancy care from medical staff. In another aspect of the present disclosure, the mobile application 120 may be further configured to include one or more links to subjects that may be of interest to the pregnant woman 115 for further educational purposes or other useful information for their pregnancy.

Often, many expectant mothers have lots of questions and sometimes they become very anxious about any change occurring in their body because they are experiencing them for the first time. Also, sometimes urgent events occur or develop such that the expectant mothers and their unborn babies need special, prompt professional attention. In such a situation, prompt communications with properly trained medical staff are key. The present technology disclosed herein provide the means or integrated platform for such communications. By way of example, based on the health data collected from the pregnant woman, the hospital or medical facility staff may desire to get in touch with the pregnant woman to discuss any developing situations or changes in her health data (e.g., abnormal data or vital data values lying outside a normal range, etc.). For example, based on review and analysis of the health data collected from the pregnant woman 115 and her fetus or unborn baby, the one or more AI agents may generate one or more alert messages and send them to the hospital or medical facility 117 or doctors or staff 119 such that they can review them and take any proactive actions or measures, such as contacting the pregnant woman 115 via various communications means including phone, the mobile device and the integrated platform. As shown in FIG. 6, the mobile application 120 on the mobile device 105 may be configured to provide such an integrated platform for communications. Furthermore, in another aspect of the present disclosure, the mobile device 105 (or the mobile application 120) may be configured to communicate with other expectant mothers in a support community, volunteers or mentors for receiving support and comfort during and after the pregnancy.

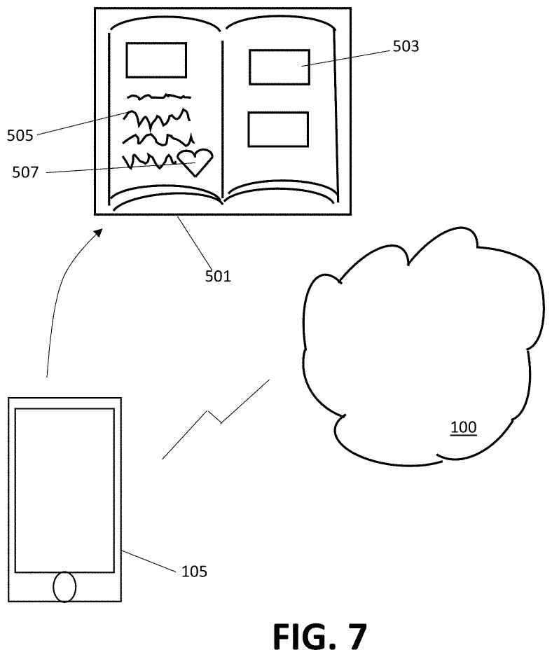

FIGS. 7-10 are diagrams conceptually illustrating one or more aspects of the present technology in various aspects of the present disclosure. By way of example, FIG. 7 shows a diagram conceptually illustrating an exemplary embodiment of a digital record book for pregnancy. In an aspect of the present disclosure, the mobile device 105 may be configured to build and generate a digital pregnancy record book 501, which documents and records various events of the pregnancy and may become part of a baby record book later. In one example, the pregnant woman 115 may desire to have records of her pregnancy by taking pictures, and in such as case, she can take photos of herself or with family members at various times, dates and locations, during her pregnancy. In particular, photos or pictures may be taken at the first time of knowledge of having a baby, at a first doctor's visit, at the time of first noticeable movement of the baby, at any other times of special memory, and so forth. Then, the photos or pictures may become part of the digital pregnancy record book 501, based on a preselected template. Further, the expectant mother may desire to include video or audio recording in the digital pregnancy record book 501. In such a case, video recordings or audio recordings or special writings may be made by the pregnant woman 115 for special moments and stored in the mobile device 105, which become part of the digital pregnancy record book 501 based on the preselected template.

In an aspect of the present disclosure, the mobile device 105 may be further configured to tag these photos, images, audio recordings, or writings with the time and dates and collage them together and make them part of the digital pregnancy record book 501, based on a template or preformatted form. The template or preformatted form for the digital pregnancy record book 501 may be selected or chosen by the pregnant woman 115 when presented by the mobile device 105. Further, one or more templates for the digital pregnancy record book 501 may be downloaded from a website and customized for making the pregnancy record book 501. Also, in the design of the digital pregnancy record book 501, one or more AI agents (installed on the mobile device 105 or cloud network) may be used based on the analysis of photo, images 503, audio recordings 507, and/or writings 505. Also, the one or more AI agents may select various layout options for each page of the digital pregnancy record book 501. In another aspect of the present disclosure, the digital pregnancy record book 501 may be built based on a profile of the pregnant woman 115. The profile of the pregnant woman 115 may include information such as age, taste, likes or dislikes, ethnic groups, culture, interests, or other user selected options.

In another aspect of the present disclosure, when the pregnant woman writes texts 505, e.g., poems for the baby, letters, essays, etc. on the mobile device 105, the one or more AI agents may convert those texts into audio recordings using the voice of the pregnant woman 115. The conversion may be done based on the natural language processing tools such that the digital pregnancy record book 501 may include the mother's natural voices and all the transformed or converted audios are available in the pregnant woman's natural voice, not using computer voices, which are often available by default.

Alternatively, when the voice or audio recordings of the pregnant woman are available, the voice or audio recordings may be converted into texts by the one or more AI agents on the mobile device 105. As noted above, based on the profile of the pregnant woman, the one or more AI agents may select one or more templates for entry into the digital pregnancy record book 501 such that its background and colors as well as its layout of each page of the digital pregnancy record book 501 may be customized. Also, the one or more AI agents may be configured to assist the pregnant woman 115 in the design process of the digital pregnancy record book 501. Also, when the publication of the digital pregnancy record book 501 is desired, the digital pregnancy record book 501 may be digitally published using the cloud platform 111 or through other third-party services, e.g., for sharing it with other people later. Alternatively, the digital pregnancy record book 501 may be published as a print album as well as an electronic version through various services that may be available on the mobile device 105.

As such, the digital pregnancy record book 501 may be configured to collage different photos, texts and audio recordings to document excitements, wonders, worries, anticipations, and memories during the pregnancy as well as the baby's development, as unique and sentimental ways in a timeline, organized format according to the date. That is, during each trimester period of the pregnancy, various events or moments may be captured and documented, which become part of the digital pregnancy record book 501 such that the pregnant woman as well as others may review them as memorable experiences and for the purpose of documenting the pregnancy of their babies.

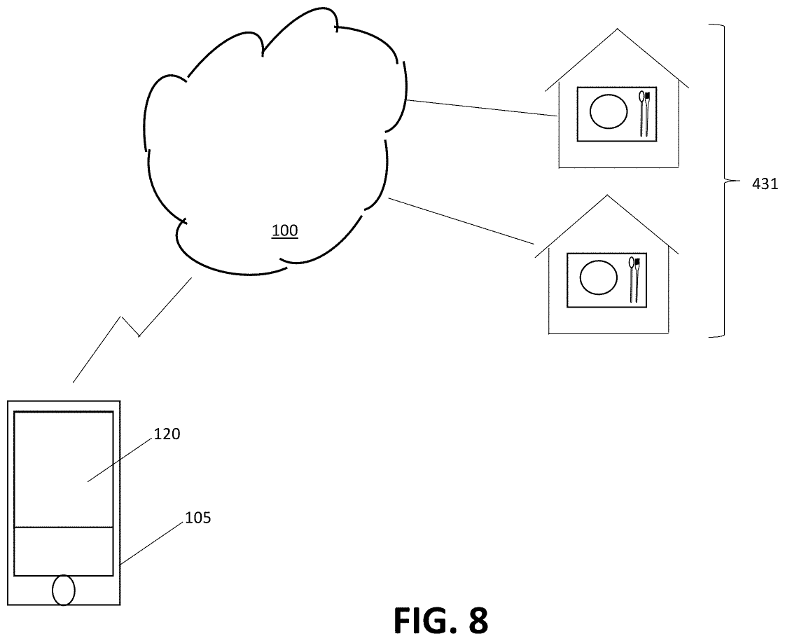

FIG. 8 shows a diagram conceptually illustrating another embodiment of the present technology in an aspect of the present disclosure. During the pregnancy, preparing meals for a pregnant woman can be a challenge for herself and others including family members. Sometimes, the pregnant woman may have a strong desire for specific foods or diets that she normally does not eat and may eat more amount of food than usual. It all depends upon the individual pregnant woman's situation and each woman may have different experiences. In an aspect of the present disclosure, the integrated platform including the mobile device 105 may provide the pregnant woman 115 with various options for foods and/or meals. Based on the health data collected by the mobile device 105 from the pregnant woman 115, the mobile device 105 may provide enhanced feature or function that is designed to help the pregnant woman to deal with such situation. That is, the mobile device 105 may be configured to analyze the health data obtained from the pregnant woman 115 and based on a profile of the pregnant woman 115, the mobile device 105 may be configured to design and present one or more recommendations for the meals and/or diet schedules, which may be provided by nearby restaurants and their menus. This is a personalized food delivery service based on specific requirements and personal preferences of the pregnant woman 115. In one implementation, the one or more AI agents may search for one or more restaurants and their menus which participate in a networked food delivery service.

By way of example, the mobile application 120 of the mobile device 105 may include a component or feature that is designed to display a variety of choices for meals that pregnant woman can order on the online integrated platform for personalized food delivery service. Based on the profile of the pregnant woman 115 and her health conditions including her weight and other vital statistics, the one or more AI agents may determine the best nutritional content, calories, and the types of meals that can be consumed by the pregnant woman 115 during a specific trimester period. Upon selection of meals, the meals may be prepared and served by one or more local restaurants 431 or may be specially designed by other places participating in the personalized food delivery service. The one or more local restaurants 431 may enter into agreements with the integrated platform or the personalized food delivery service, for preparing and delivering the meals with proper nutritional content according to specific, personalized requirements of the pregnant woman 115. For example, the one or more AI agents may design, search, or recommend meals that are designed to prevent development of diabetic conditions of the pregnant woman based on the collected health data of the pregnant woman, but also help and maintain healthy physical well-being of the pregnant woman. As such, the present technology disclosed herein, including the integrated platform, may design and provide a personalized meal service for expectant mothers, based on her health data and the personal profile of the pregnant woman 115 (e.g., age, weight, preferences, ethnic backgrounds, dietary recommendations, pre-existing health conditions, etc.) and the one or more recommendations made by the hospital or medical facility 117.

As mentioned above, based on the profile of the pregnant woman 115, the one or more AI agents may search, design and recommend a variety of choices for meals during and after her pregnancy. On the mobile device 105, after viewing a variety of choices for each meal suggested by the one or more AI agents, the pregnant woman 115 may select and order the meal. Further, the one or more AI agents may be configured to track certain information on the content, calories, and nutritional values of the meals ordered and consumed by the pregnant woman 115 and store the data for analytics purposes, which may become part of pregnancy data of the woman.

In another aspect of the present disclosure, the mobile device 105 or the one or more AI agents may also recommend one or more specially prepared meals for the woman after the child-birth of a baby, to help quickly recover from the delivery of the baby, based on her profile and related health data. In this case, the mobile device 105 may be configured to perform as a broker for sales of meals between the local restaurants 431 and the users of the mobile device 105.

In another aspect of the present disclosure, the users of the mobile device 105 may take pictures or photos of a prepared meal and based on the image processing of the pictures or photos, the mobile device 105 may identify each food component of the meal and determine calorie amounts and other dietary data and display them to the user either in real-time or offline formats. Upon review of the displayed data including the meal content and calories, other dietary data, the pregnant woman 115 may review and consume the entire meal or select and consume a few items of the meal to meet her personal calorie intake goal or desire. Further, the one or more AI agents may be configured to suggest different combinations of meal items for consumption by the pregnant woman 115, based on past food consumption data, during different stages of the trimester period of the pregnancy.

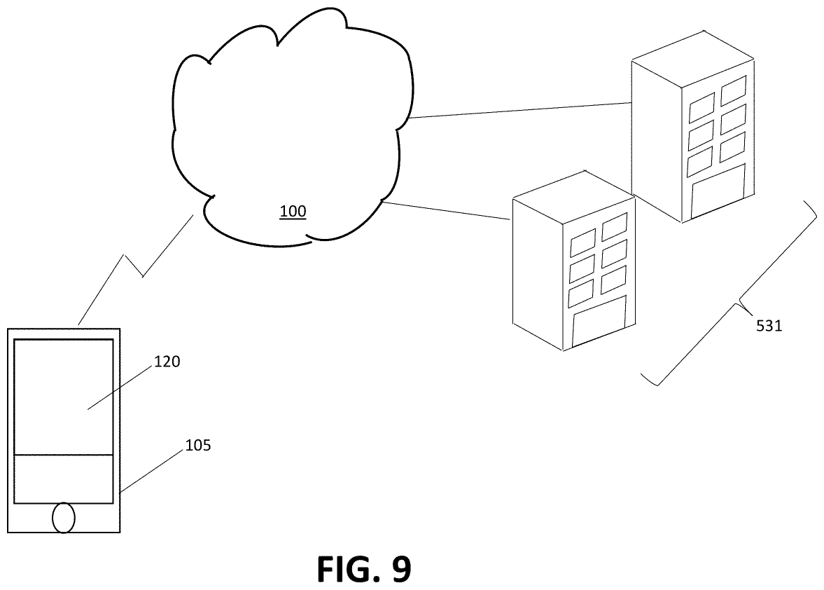

FIG. 9 shows another diagram conceptually illustrating an exemplary embodiment of the present technology in accordance with an aspect of the present disclosure. After the childbirth, mothers often need good care and resting for recovery. Although there are many variations in terms of different types of post-delivery care at the hospital or medical facility, women often stay at the hospital bed for 2-3 days and discharged to home for care, which is just too short for full recuperation. This short recovery time is often not enough for numerous mothers who are exhausted after intensive labor pain and delivery. Thus, many mothers may need good care and resting away from her newborn baby and as a result the mother or family members often make an additional arrangement for post-delivery care options at different facilities. At the post-delivery care facilities outside the hospital or medical facility, mothers may receive needed full care and resting for quick recovery and recuperation from the intensive labor pain or childbirth event.

In most cases, the child-birth event is a very significant, life changing event for mothers, directly affecting the well-being and health of the mothers, and as such, many women find that they need to recover well from the event, physically, emotionally, and spiritually. However, the additional stay at the hospital after the child-birth event is very limited in time duration, e.g., 2-3 days at most, before discharge from the hospital. Upon discharge from the hospital, mothers either return home or go to a third party care facility (e.g., post-delivery care facility) for receiving post-delivery care and resting for an additional period of time. The post-delivery care facilities are facilities with medical help designed to provide the women with needed post-delivery care and resting such that they can return back to normal life.

In an aspect of the present disclosure, the present technology disclosed herein provides a means for providing one or more options for the post-delivery care facilities. By way of example, the mobile application 120 of the mobile device 105 may be configured to serve as an integrated platform by presenting one or more options about these post-child delivery care facilities for new mothers. The one or more options may be based on the profile of the pregnant woman or user input such as preferences, locations, costs, durations, etc. As part of the integrated platform, based on the profile and the additional information, the mobile application 120 may prepare and present to the user a variety of choices relating to the third party post-delivery care facility for the purpose of recovering from the intensive child delivery at the hospital. In the example, the mobile application 120 of the mobile device 105 may be configured to gather information on post-delivery care facilities near homes of new mothers and present to the new mothers, via one or more interfaces of the mobile application 120, such that the new mothers can browse and select one of the post-delivery care facilities. The information displayed about the post-delivery care facilities may include information on the facilities, such as post-delivery duration, costs, locations, conditions, services, facility profiles including photos, audio and video comments or reviews, comments by previous users of the post-delivery care facilities, and so forth. As such, when planning for the post-delivery care, the user of the mobile application 120 may browse, search and make an arrangement for the transition from a delivery facility (or hospital) to a post-delivery care facility in a smooth and efficient manner.

As such, in one implementation, when a due date for child-birth is close, the one or more AI agents may automatically prepare and display the options for the post-delivery care facilities to the user via either a separate window or as part of the additional gadget window in the mobile application 120 of the mobile device 105. Also, in another aspect of the present disclosure, the one or more AI agents of the mobile device 105 in cooperation with the mobile application 120 may act as a broker for transactions between the post-delivery care facility and the user of the mobile device 105.

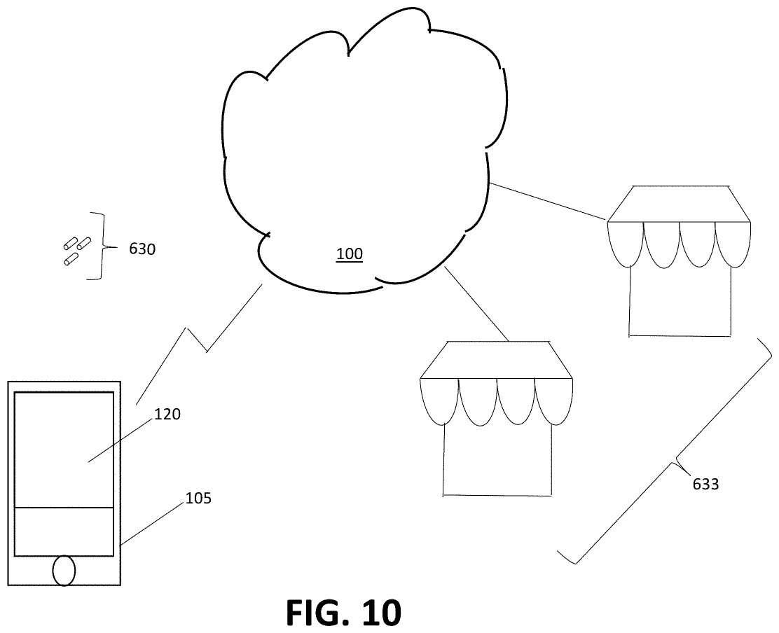

Further, in another aspect of the present disclosure, the mobile application 120 of the mobile device 105 may serve as a platform for transacting sales of prenatal, pregnancy and post natal supplements to pregnant women. FIG. 10 shows a diagram conceptually illustrating another embodiment of the present technology in an aspect of the present disclosure. The mobile application 120 may include or integrate one or more platforms or applications for online shopping for pre-natal or post-natal care supplement products 630 for use by pregnant women. By way of example, in the mobile application 120, the pre-natal care supplements may be displayed with customer ratings and presented with ranking information to the pregnant woman such that the pregnant woman can review, select, and purchase the supplement products 630 sold or distributed by various stores 633. Further, the mobile application 120 may be configured to display all relevant medical information relating to the supplement products sold or distributed by various stores 633.

In another aspect of the present disclosure, the one or more AI agents may use the collected health data of the pregnant woman 115 to make a list of recommendations for the types of pre-natal supplement products 630 that may be essential and helpful for improving and maintaining the health and good well-being of the pregnant woman 115.

Also, the one or more AI agents may be further configured to track and/or monitor daily consumption of the pre-natal supplement products 630 and any change in the health data of the pregnant women 115 during their pregnancy such that in case of an abnormality is found one or more alert messages are generated and sent to the hospital or medical facility for follow-up review and action. That is, the one or more AI agents may be configured to generate one or more alarms to doctors or staff at the hospital when certain changes are detected in the health data of the pregnant woman 115 while she is taking any of the recommended supplement products 630 during her pregnancy. In such a case, the doctors or staff at the hospital 117 will review the health data of the pregnant woman 115 and make any recommended adjustments or changes in the consumption of supplements, foods or other medications to mitigate the situation. Furthermore, the one or more AI agents may be designed to enable the pregnant woman 115 to take more proactive actions based on the analysis of the collected health data of the pregnant woman 115. That is, the one or more AI agents may be configured to determine appropriate amounts of supplements and intake schedules to optimize the use of the supplements 630 during the pregnancy, based on the collected health data related to the pregnant woman 115 as well as the unborn baby of the pregnant woman 115.

Furthermore, in another aspect of the present disclosure, the integrated platform with the mobile device 105 may provide easy access to a set of educational resources relating to the pregnancy, serving as a single access point for new mothers. The educational resources may be available in various formats including videos, YouTubes, audios, texts, etc. and may be presented by the integrated platform in an organized, easy to access manner designed by the one or more AI agent, based on the profile of the pregnant woman and health data. Also, the educational topics may include resources available for emotional support and educational materials for the pregnancy, new mother's roles, new father's roles, emotional support during the pregnancy as well as labor, prenatal care, information on the first trimester, second trimester, third trimester, fourth trimester, and child-birth, and information on high risk pregnancy, complications and symptoms, etc. As such, the mobile device 105 or the mobile application 120 may serve as the single point of access for all the necessary resources.

As such, for at least the foregoing, various aspects of the present technology disclosed herein provide numerous benefits and means to pregnant women in general and help them ease with the pregnancy as well as reduce the chance of having late response to any complication situation by providing enhanced means for having timely, effective communications between the hospital or medical facilities and the pregnant women during their pregnancy, based on the collected health data from the pregnant women and their unborn babies.

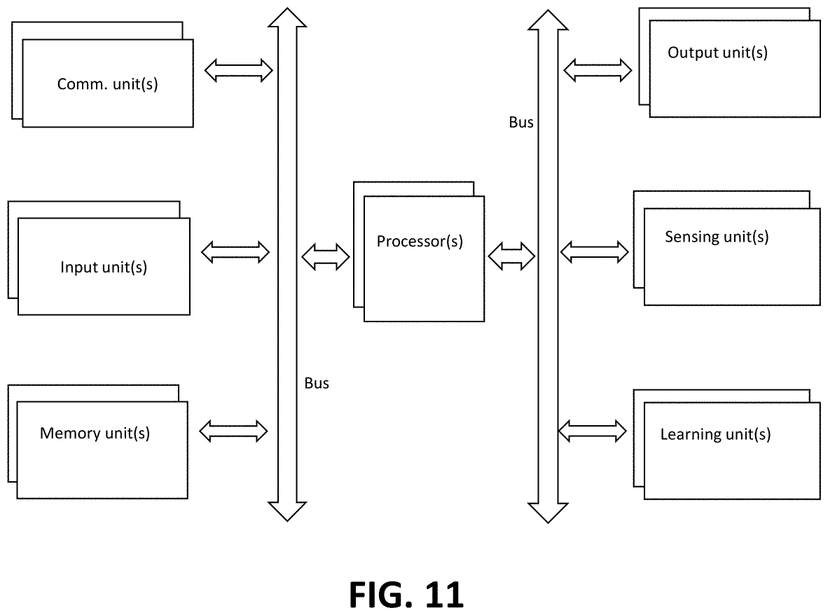

In an aspect of the present disclosure, the mobile device or other electronics devices may be implemented by one or more processing system(s). FIG. 11 is a diagram conceptually illustrating an example of a processing system in accordance with an aspect of the present disclosure. By way of example, an apparatus of FIG. 11 may be implemented in or part of various computing devices including the mobile device and wearable devices, such as the first wearable device, the second wearable device, and the electronic scale, as shown in various figures.

By way of example, the embodiment of FIG. 11 may include one or more communication unit(s), one or more input unit(s), one or more memory unit(s), one or more output unit(s), one or more processor(s), one or more sensing unit(s), and one or more learning unit(s), all of which are configured to communicate with each other via one or more bus(s). The one or more processor(s) may be configured to perform artificial processing through one or more AI agents, along with the learning unit(s).

In the example, the communication unit(s) are configured to transmit and receive data to and from one or more external devices via various communications technology, including but not limited thereto, such as wired and/or wireless communication technologies, e.g., WiFi, Ethernet, fifth generation (5G), six generation (6G), Internet of Things (IoT), wireless local area network (LAN), Bluetooth, radio frequency identification (RFID), near field communications (NFC) and the like. The input unit(s) may be configured to include one or more sensors, cameras, microphones, and/or user inputs. The input unit(s) may also be configured to acquire new raw data based on features extracted from the data processed by the learning unit(s).

In the example of FIG. 11, the bus may be configured to link together various components or circuits, including the one or more processors (or processing systems), one or more memories (or memory unit(s)), one or more communication units (or comm unit(s), and/or one or more input/output devices (or input unit(s) and output unit(s)). The one or more processing systems may be responsible for managing the bus and general processing, including the execution of software stored on a non-transitory computer-readable medium. Also, the one or more processing systems may include one or more processors, such as microprocessors that interpret and execute instructions.

In other implementations, the one or more processing systems may be implemented in or include one or more application specific integrated circuits (ASICs), field programmable logic arrays, or the like. Also, the software, when executed by the one or more processing systems, may cause the one or more processing systems to perform the various functions described herein for any particular apparatus. The non-transitory computer-readable medium may also be used for storing data that is manipulated by the one or more processing systems when executing software. The one or more memories may include various types of memories, including a random-access memory and/or a read only memory, and/or other types of magnetic or optical recording medium and its corresponding device for storing information and/or instructions and/or retrieval thereof. The one or more communication units may also include any transceiver-like mechanism that enables communication with other devices and/or systems. The one or more input/output devices may include devices that permit inputting information and/or outputting information to an operator.

In another aspect of the present disclosure, the learning unit(s) may be configured to include one or more memory unit(s) integrated or integrated in the device or apparatus, or externally connected to the apparatus. Further. the learning unit(s) may also be configured to learn one or more models, each of which may comprise an artificial neural network by using learned data. As mentioned, the learning unit(s) may work with one or more AI agents described herein. In the example, such a learned artificial neural network may be referred to as a learning model. The learning model may be used to infer a value for new input data rather than learning data and the inferred value may be used as a basis for determining to perform a certain operation. Further, the learning model may be built and matured through various artificial intelligence techniques including deep learning techniques as well as others. In the example, the learning model may be matured through operations of the one or more AI agents disclosed herein.

In another aspect of the present disclosure, in one implementation, the mobile device as shown in FIGS. 1 and 2 may be configured to work together with one or more learning models independently or in conjunction with the one or more AI agents. The one or more learning models may be built based on collected data from wearable devices or wearable sensors. The one or more learning models may thus be part of software and/or hardware components of the mobile device.

In an aspect of the present disclosure, the sensing unit(s) may be configured to acquire data from various sensors including wearable devices and patches. The output unit(s) may be configured to generate an output relating to a visual sense, an auditory sense, or the like and may be configured to include one or more display units(s) for outputting various pieces of information. The memory unit(s) may be configured to store data for various functions, for example, input data acquired by the input unit(s) or sensing unit(s), learning data, learning models, learning history, and the like.

In another implementation, as mentioned above, the mobile device may learn about a patient or pregnant woman through various sensors or devices. A large set of health data may be collected and built into one or more artificial neural networks to form and tune the one or more learned models or the one or more AI agents. The one or more learned models can then be used to automatically generate alerts or take proactive steps to mitigate any risk to a user, patient, pregnant woman, hospital, medical facility, doctors, or medical professional, or the like. Further, the one or more learned models may be further enhanced by means of communications with other remote devices disposed at one or more medical facilities over a cloud-based network.

Even though particular combinations of features are disclosed in the specification and/or recited in the claims, these combinations are not intended to limit the disclosure of the present technology. Further, various aspects of the present technology disclosed herein may be implemented in software, hardware, any combinations of software and hardware, a computer program or firmware incorporated in a computer readable medium for execution by a controller, a processor, a computer, or a processing system that includes one or more processors. Examples of processors include microcontrollers, microprocessors, digital signal processors (DSPs), discrete hardware circuits, gated logic, state machines, programmable logic devices (PLDs), field programmable gate arrays (FPGAs), and other suitable hardware configured to perform various functions described herein.

These various implementations may include implementations in one or more computer programs that are executable and/or interpretable on one or more programmable systems including at least one programmable processor coupled to receive data and instructions for a programmable processor and may be implemented in a high-level procedural and/or object-oriented programming language, and/or in assembly or machine language. The term “machine-readable medium” and “computer-readable medium” as used herein refer to any computer program product(s), apparatus and/or device used to provide machine instructions and/or data to a programmable processor, including a machine-readable medium that receives machine instructions as a machine-readable signal. Also, the term “software” as used herein includes various types of machine instructions including instructions, code, programs, subprograms, software modules, applications, software packages, routines, subroutines, executables, procedures, functions, etc. The software may also refer to general software, firmware, middleware, microcode, hardware description language, or etc. As noted above, the software may be stored on a computer-readable medium.

Examples of a computer-readable medium may include a non-transitory computer-readable medium, such as, by way of example, an optical disk, a magnetic storage device, a digital versatile disk, a flash memory, random access memory (RAM), read only memory (ROM), a register, programmable ROM (PROM), erasable PROM (EPROM), electrically erasable PROM (EEPROM), a removable disk, a flash memory device, and any other suitable medium for storing software that may be accessed and read by a processor or a processing system. It is also appreciated that one skilled in the art will recognize how best to implement the described functionality relating to adding new system functionalities to an existing network element, depending upon a particular application within design constraints.

The term “unit,” “device” or “component” as used herein means software, hardware, or any combinations thereof. A unit may be implemented as a software component, a hardware component, or any combinations thereof, including a FPGA, logic, logic arrays, ASIC, DSP, microcontroller, microprocessor, etc. or any combinations thereof. The unit thus may include software components, task components, processes, procedures, functions, program code, firmware, micro-codes, circuits, data structures, tables, arrays, and variables.

The term “article of manufacture” is intended to encompass a computer program accessible from any computer-readable device, carrier, or medium. A singular form may include a plural form if there is no clearly opposite meaning in the context.

As shown above, various methods, techniques, arrangements or their variants may be implemented for artificial trees or plants with capability of producing scent or other features. Other embodiments of the present technology may be possible and are not limited to the disclosed embodiments herein.

Further, as mentioned above, it is noted that as used in the specification and the appended claims, the singular forms “a”, “an”, and “the” include plural referents or one or more items, unless the context clearly indicates otherwise. Also, no element, act, step, or instruction used in the present disclosure should be construed as critical or essential to the present disclosure unless explicitly described as such in the present disclosure. As used herein, except explicitly noted otherwise, the term “comprise” or variations of the term, such as “comprising,” “comprises,” and “comprised” are not used to exclude other additives, components, integers or steps. The term “first,” “second,” and so forth used herein may be used to describe various components, but the components are not limited by the above terms. The above terms are used only to discriminate one component from other components, without departing from the scope of the present disclosure. Also, the term “and/or” as used herein includes a combination of a plurality of associated items or any item of the plurality of associated items. Further, it is noted that when it is described that an element is “coupled” or “connected” to another element, the element may be directly coupled or directly connected to the other element, or the element may be coupled or connected to the element through a third element. Also, the term “include” or “have” as used herein indicates that a feature, an operation, a component, a step, a number, a part or any combination thereof described herein is present. Furthermore, the term “include” or “have” does not exclude a possibility of presence or addition of one or more other features, operations, components, steps, numbers, parts or combinations. It is also noted that the foregoing relates only to exemplary embodiments of the present invention or technology and that numerous modifications or alternations may be made therein without departing from the spirit and the scope of the present disclosure as set forth in this disclosure.

Although the exemplary embodiments of the present disclosure are provided herein, the present disclosure is not limited to these embodiments. There are numerous modifications or alternations that may suggest themselves to those skilled in the art. It is appreciated by one skilled in the art that a wide variety of alternate and/or equivalent implementations may be substituted for the specific examples shown and described without departing from the scope of the present disclosure. As such, the exemplary embodiments should not be construed in any way as imposing limitations upon the scope thereof. One the contrary, it is understood that various other embodiments, modifications, and equivalents thereof which, after reading the description herein, may suggest themselves to those skilled in the art without departing from the spirit of the present invention or disclosure and/or the scope of the appended claims.

What is claimed is:

1. An apparatus for providing health services to a pregnant woman, the apparatus comprising:
a first wearable device to be worn by the pregnant woman, the first wearable device being configured to collect a first set of health data of the pregnant woman;
a second wearable device to be worn by the pregnant woman, the second wearable device being configured to collect a second set of health data of an unborn baby growing in an abdomen of the pregnant woman; and
a mobile device configured to communicate with the first wearable device and the second wearable device,
wherein the mobile device is configured to:
collect the health data of the pregnant woman and the unborn baby,
transmit the health data to a remote hospital over a cloud-based network including one or more servers; and
receive guidance or instructions from the remote hospital in response to the transmission of the health data of the pregnant woman and the unborn baby.

2. The apparatus of claim 1, wherein the first set of health data comprises: a heart rate, blood pressure, weight, glucose level, temperature, and/or oxygen saturation of the pregnant woman; and wherein the second set of health data comprises a heart rate, body parts, and/or size of the unborn baby.

3. The apparatus of claim 2, wherein the first wearable device comprises a smart watch or a smart band to be worn on a wrist of the pregnant woman and wherein the second wearable device comprises a smart device disposed on the abdomen area of the pregnant woman for collecting the health data of the unborn baby.

4. The apparatus of claim 1, further comprising a multi-functional electronic scale for measuring a body weight of the pregnant woman, wherein the multi-functional electronic scale is configured to wirelessly communicate with the mobile device.

5. The apparatus of claim 1, wherein the mobile device is further configured to display data analysis information to the pregnant woman via one or more user interfaces on the mobile device, based on the health data collected from the pregnant woman.

6. The apparatus of claim 1, wherein the mobile device comprises one or more artificial intelligence (AI) agents for analyzing the health data collected from the pregnant woman and presenting one or more recommendations to the pregnant woman based on the analysis of the collected health data.

7. The apparatus of claim 2, wherein the mobile device is further configured to receive one or more messages from the remote hospital when an abnormality is detected in the health data of the pregnant woman and the unborn baby.

8. The apparatus of claim 1, wherein the mobile device is further configured to automatically provide the pregnant woman with one or more recommendations for dietary meals for the pregnant woman, based on a profile and the health data of the pregnant woman and wherein the profile of the pregnant woman comprises her age, color of skin, ethnic group, pre-existing conditions, or geographical regions.

9. The apparatus of claim 1, wherein the mobile device is further configured to provide the pregnant woman with one or more recommendations for post-delivery care facilities, based on a profile of the pregnant woman and wherein the profile of the pregnant woman comprises her age, color of skin, ethnic group, pre-existing conditions, or geographical regions.

10. The apparatus of claim 1, wherein the mobile device is further configured to provide the pregnant woman with one or more recommendations for supplementary medicines, based on a profile and the health data of the pregnant woman.

11. The apparatus of claim 1, wherein the system is further configured to produce a digital record book relating to a pregnancy of the pregnant woman and wherein the digital record book comprises digital pictures, photos, audios, writings, or any combinations thereof.
